(19) United States
(12) Patent Application Publication
(10) Pub. No.: US 2024/0029860 A1
(43) Pub. Date: Jan. 25, 2024

(54) HEALTH MANAGEMENT SUPPORT SYSTEM, ITS CONTROL METHOD, AND CONTROL PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Shotaro YASUDA, Taito-ku (JP); Hiroki SATO, Chuo-ku (JP); Hiroaki KAMEI, Taito-ku (JP); Maika HASHIMOTO, Urayasu-shi (JP); Manabu YAMAMOTO, Toyota-shi (JP)

(73) Assignee: TOYOTA JIDOSHA KABUSHIKI KAISHA, Toyota-shi (JP)

(21) Appl. No.: 18/356,790

(22) Filed: Jul. 21, 2023

(30) Foreign Application Priority Data
Jul. 25, 2022 (JP) 2022-117831

(51) Int. Cl. G16H 20/60; G16H 20/30; G16H 50/30

(52) U.S. Cl. CPC G16H 20/60; G16H 20/30; G16H 50/30

(57) ABSTRACT

A health management support system includes: an acquisition unit configured to acquire information about a current state of a user, information about a target health condition of the user after a lapse of a set period, and information about a budget of the user; a plan calculation unit configured to calculate at least one plan for a behavioral item that the user should perform during the set period based on the information about the current state of the user, the target health condition of the user after the lapse of the set period, and the budget; and an output unit configured to output the at least one plan calculated by the plan calculation unit.

### CROSS REFERENCE TO RELATED APPLICATIONS

This application is based upon and claims the benefit of priority from Japanese patent application No. 2022-117831, filed on Jul. 25, 2022, the disclosure of which is incorporated herein in its entirety by reference.

### BACKGROUND

The present disclosure relates to a health management support system, its control method, and a control program.

In recent years, systems for assisting users to manage their health have been developed.

For example, Japanese Unexamined Patent Application Publication No. 2010-176242 discloses a health guidance support program for causing a CPU (Central Processing Unit) of a health guidance support system to function so as to display, in a comparative manner, a target energy reduction amount for achieving a received target of a user and an energy reduction amount that is achieved by carrying out a plan obtained for the current state of the user based on both information about the current living situation of the user and his/her life improvement plan.

### SUMMARY

However, in Japanese Unexamined Patent Application Publication No. 2010-176242, the health guidance support system merely displays, in the comparative manner, the target energy reduction amount for achieving the target set by the user and the energy reduction amount that is achieved by carrying out the life improvement plan set by the user, but does not propose a life improvement plan (a behavioral plan) suitable for achieving the target set by the user. That is, in Japanese Unexamined Patent Application Publication No. 2010-176242, the health guidance support system does not propose an appropriate behavioral plan for bringing the health condition of the user closer to his/her target health condition.

The present disclosure has been made in view of the above-described circumstances, and an object thereof is to provide a health management support system, its control method, and a control program capable of proposing an appropriate behavioral plan for bringing the health condition of a user closer to his/her target health condition.

A health management support system according to an embodiment of the present disclosure includes: an acquisition unit configured to acquire information about a current state of a user, information about a target health condition of the user after a lapse of a set period, and information about a budget of the user; a plan calculation unit configured to calculate at least one plan for a behavioral item that the user should perform during the set period based on the information about the current state of the user, the target health condition of the user after the lapse of the set period, and the budget; and an output unit configured to output the at least one plan calculated by the plan calculation unit. The above-described health management support system can propose an appropriate behavioral plan for bringing the health condition of the user closer to his/her target health condition before a set period expires by taking not only the information about the current state of the user (such as the current health condition) and the target health condition of the user after the lapse of the set period but also the budget of the user into consideration.

The information about the current state of the user may include information about a current health condition of the user. The current health condition of the user is, for example, the current body shape of the user and/or the current body fat percentage thereof.

The information about the current state of the user may include information about at least one of a current age, a gender, a current height, and a current weight of the user.

The information about the current state of the user may include information about at least one of a currently preferred food of the user and a currently preferred exercise thereof. It is possible to motivate the user to carry out his/her behavioral plan by incorporating the preferred food and/or preferred exercise into the plan.

The information about the current state of the user may include at least one of information about a food that the user is currently prohibited from ingesting and information about an exercise that the user is currently prohibited from doing. In this way, for example, the plan is calculated so that any food that may cause an allergic reaction is not used therein.

The behavioral item of the user may include at least one of an exercise item and a meal.

The plan calculation unit may: extract a behavioral item and an improvement in a health condition, based on a selected plan, of at least one past subject of which, among behavioral items and improvements in health conditions, based on the selected plan, of a plurality of past subjects registered in a first database, the information about the state at that time, the target health condition after the lapse of the set period, and the budget have been determined to be similar to the information about the current state, the target health condition after the lapse of the set period, and the budget of the user; and calculate at least one plan for a behavioral item that the user should perform during the set period by referring to the behavioral item and the improvement in the health condition, based on the selected plan, of the extracted past subject.

The plan calculation unit may also calculate at least one new plan for the behavioral item that the user should perform during the set period based on a new budget that is obtained by adding a predetermined amount of money to the budget, and the output unit may also output the at least one new plan calculated by the plan calculation unit along with information about the new budget.

The plan calculation unit may calculate, in addition to the information about the current state of the user, the target health condition of the user after the lapse of the set period, and the budget, at least one plan for the behavioral item that the user should perform during the set period based on a schedule of the user during the set period stored in a second database.

The output unit may display one of selected plans among the at least one plan calculated by the plan calculation unit on a display unit along with the schedule of the user.

A method for controlling a health management support system according to an embodiment of the present disclosure includes: acquiring information about a current state of a user, information about a target health condition of the user after a lapse of a set period, and information about a budget; calculating at least one plan for a behavioral item that the user should perform during the set period based on the information about the current state of the user, the target health condition of the user after the lapse of the set period, and the budget; and outputting the at least one plan calculated by the plan calculation unit. By the above-described method for controlling a health management support system, it is possible to propose an appropriate behavioral plan for bringing the health condition of the user closer to his/her target health condition before a set period expires by taking not only the information about the current state of the user (the current health condition or the like) and the target health condition of the user after the lapse of the set period but also the budget of the user into consideration.

A control program according to an embodiment of the pre sent disclosure causes a computer to perform: a process for acquiring information about a current state of a user, information about a target health condition of the user after a lapse of a set period, and information about a budget of the user; a process for calculating at least one plan for a behavioral item that the user should perform during the set period based on the information about the current state of the user, the target health condition of the user after the lapse of the set period, and the budget; and a process for outputting the at least one plan calculated by the plan calculation unit. By the above-described control program, it is possible to propose an appropriate behavioral plan for bringing the health condition of the user closer to his/her target health condition before a set period expires by taking not only the information about the current state of the user (the current health condition or the like) and the target health condition of the user after the lapse of the set period but also the budget of the user into consideration.

According to the present disclosure, it is possible to provide a health management support system, its control method, and a control program capable of proposing an appropriate behavioral plan for bringing the health condition of a user closer to his/her target health condition.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a health management support system according to a first embodiment;

FIG. 2 is a flowchart showing operations performed by the health management support system shown in FIG. 1;

FIG. 3 shows an example of input items displayed on a monitor;

FIG. 4 shows an example of behavioral plans displayed on a monitor;

FIG. 5 shows another example of behavioral plans displayed on a monitor; and

FIG. 6 shows an example of a behavioral plan displayed on a monitor along with a schedule of a user.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

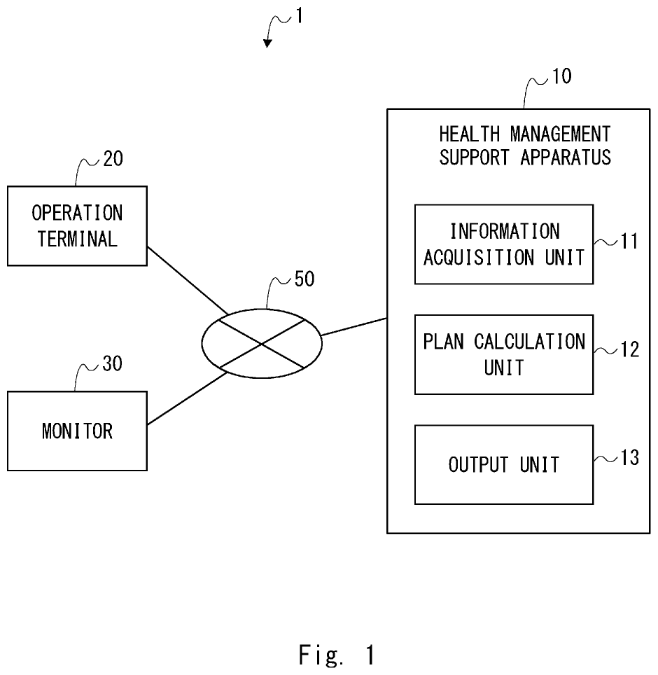

FIG. 1 is a block diagram showing an example of a configuration of a health management support system 1 according to a first embodiment. The health management support system 1 can propose an appropriate behavioral plan for bringing the health condition of the user closer to his/her target health condition before a set period expires by taking not only the information about the current state of the user and the target health condition of the user after the lapse of the set period but also the budget of the user into consideration. Details of the health management support system 1 and the like will be described hereinafter.

As shown in FIG. 1, the health management support system 1 includes a health management support apparatus 10, an operation terminal 20, a monitor 30, and a network 50. The health management support apparatus 10 can also be referred to as a health management support system. The operation terminal 20, the monitor 30, and the health management support apparatus 10 are configured so that they can communicate with each other through the wired or wireless network 50.

The operation terminal 20 is, for example, a PC (Personal Computer) terminal, a mobile terminal such as a smartphone or a tablet-type terminal, or a dedicated communication terminal provided for the body shape management system. The operation terminal 20 receives information about the current state of the user U1, information about a target health condition of the user U1 after a lapse of a set period, and information about a budget of the user, entered by an operation performed by the user U1, and transmits the received information items to the health management support apparatus 10 through the network 50.

Note that the information about the current state of the user U1 includes, for example, information about at least one of the current age, the gender, the current height, and the current weight of the user U1. Further, the information about the current state of the user U1 may include information about the current health condition of the user U1. The information about the current health condition of the user U1 is, for example, the current body shape of the user U1 and/or the current body fat percentage thereof. Note that the information about the current body shape and the current body fat percentage of the user U1 may be directly entered by the user U1, or may be estimated by the operation terminal 20 or the health management support apparatus 10 based on the current height, the current weight, and the like of the user U1.

Further, the information about the current state of the user U1 may include information about at least one of a currently preferred food of the user U1 and a currently preferred exercise thereof. As the preferred food, foods that are roughly classified, such as being classified into fish, meat, vegetables, and the like, may be designated, or foods that are finely classified, such as being classified, among various types of fish, into pike, salmon, eel, and the like, may be designated. Alternatively, as the preferred food, a specific meal menu using a specific food(s) may be designated. As the preferred exercise, for example, running, rope jumping, swimming, tennis, or a training in a gym (e.g., a strength training using a machine) is designated.

Further, the information about the current state of the user U1 may include at least one of information about a food that the user U1 is currently prohibited from ingesting and information about an exercise that the user is currently prohibited from doing. As the food that the user is prohibited from ingesting, foods that are roughly classified, such as being classified into fish, meat, vegetables, and the like, may be designated, or foods that are finely classified, such as being classified, among various types of fish, into pike, salmon, eel, and the like, may be designated. More specifically, as the food that the user is prohibited from ingesting, eggs, milk, wheat, or buckwheat, which may cause a food allergy, may be designated. As the exercise that the user is prohibited from doing, running, rope jumping, swimming, tennis, or training in a gym (e.g., a strength training using a machine) is designated. More specifically, as the exercise that the user is prohibited from doing, an exercise in which the user uses a part of the body of which the movement is restricted due to a fracture of a bone or fatigue, e.g., playing catch in a state in which his/her shoulder is fatigued, is designated.

The information about the target health condition of the user U1 after the lapse of the set period is, for example, a target body shape, a target weight, or a target body fat percentage of the user U1 after the lapse of the set period. The set period can be arbitrarily set by the user U1, and can be set to, for example, one month, three months, or one year.

The information about the budget is information about an amount of money that the user U1 can spend to carry out the behavioral plan calculated by the health management support apparatus 10 (which will be described later).

The monitor 30 is, for example, a monitor of a PC terminal, a monitor of a mobile terminal such as a smartphone or a tablet-type terminal, or a dedicated monitor prepared for the body shape management system. The monitor 30 receives results output from the health management support apparatus 10 (which will be described later) through the network 50, and displays the received results.

Note that the monitor 30 may be integrated into the operation terminal 20 or provided separately from the operation terminal 20. In this embodiment, an example case where the monitor 30 is integrated into the operation terminal 20 will be described. That is, in this embodiment, an example case where the monitor 30 is used as the monitor of the operation terminal 20, which is a PC terminal, will be described. Therefore, in addition to the results output from the health management support apparatus 10, input items for information about the current state of the user U1 or the like may be displayed on the monitor 30. In this case, the user U1 enters necessary information in input items displayed on the monitor 30 by operating a keyboard or the like of the operation terminal 20, which is a PC terminal, and thereby enters information about the current state of the user U1, information about the target health condition of the user U1 after the lapse of the set period, and information about the budget of the user into the operation terminal 20. The operation terminal 20 receives these information items entered by the operation performed by the user U1, and transmits the received information items to the health management support apparatus 10 through the network 50.

The health management support apparatus 10 calculates, based on the information about the current state of the user U1, the target health condition of the user U1 after the lapse of the set period, and the budget, at least one plan (one behavioral plan) for behavioral items that the user U1 should perform during the set period, and outputs the calculated plan.

Specifically, the health management support apparatus 10 includes at least an information acquisition unit 11, a plan calculation unit 12, and an output unit 13.

The information acquisition unit 11 acquires information entered by the user U1 through the operation terminal 20. That is, the information acquisition unit 11 acquires, for example, information about the current state of the user U1, information about the target health condition of the user U1 after the lapse of the set period, and information about the budget. Note that the information acquired by the information acquisition unit 11 is not limited to the above-described information items. That is, the information acquisition unit 11 may acquire other information used for the calculation of a behavioral plan performed by the plan calculation unit 12 (which will be described later).

The plan calculation unit 12 calculates, based on the information about the current state of the user U1, the target health condition of the user U1 after the lapse of the set period, and the budget, acquired by the information acquisition unit 11, at least one plan (one behavioral plan) for behavioral items that the user U1 should perform during the set period. Note that the behavioral items include meals and exercise items.

For example, as the information about the current state of the user U1, the current weight of the user U1 is set to 80 kg. Further, regarding the target health condition of the user U1 after the lapse of the set period, the set period is set to three months and the target weight of the user U1 after the lapse of the set period is set to 70 kg. Furthermore, the budget is set to 50,000 yen. In this case, the plan calculation unit 12 calculates at least one behavioral plan including meals and exercise items for reducing the weight of the user U1 from 80 kg to 70 kg in three months.

Note that when a preferred food of the user U1 is designated, the plan calculation unit 12 calculates a behavioral plan including a meal menu using the preferred food of the user U1 as long as it is within the budget and as long as the target can be achieved. Alternatively, when the preferred exercise item of the user U1 is designated, the plan calculation unit 12 calculates a behavioral plan including the preferred exercise item of the user U1 as long as it is within the budget and as long as the target can be achieved.

Further, when a food that the user U1 is prohibited from ingesting is designated because of a possibility of an occurrence of a food allergy or the like, the plan calculation unit 12 calculates a behavioral plan in which the meal menus include only those in which this food is not used. Alternatively, when an exercise item that the user U1 is prohibited from doing is designated because of a fracture of a bone or fatigue, the plan calculation unit 12 calculates a behavioral plan in which the exercise items include only those other than this exercise item.

Note that the plan calculation unit 12 may calculate, based on the information acquired by the information acquisition unit 11, a behavioral plan in which conditions for exercise items are made stricter, instead of relaxing conditions for meals. For example, the plan calculation unit 12 may calculate a behavioral plan in which calories to be consumed by exercises are increased, instead of relaxing the restriction on calories taken by meals.

Alternatively, the plan calculation unit 12 may calculate, based on the information acquired by the information acquisition unit 11, a behavioral plan in which conditions for meals are made stricter, instead of relaxing conditions for exercise items. For example, the plan calculation unit 12 may calculate a behavioral plan in which the restriction on calories taken by meals is made stricter, instead of reducing calories to be consumed by exercises.

The output unit 13 outputs at least one behavioral plan calculated by the plan calculation unit 12. The information about at least one behavioral plan output from the output unit 13 is transferred, for example, to the monitor 30 through the network 50, and is displayed on the monitor 30.

As described above, the health management support system 1 according to this embodiment can propose an appropriate behavioral plan for bringing the health condition of the user U1 closer to his/her target health condition before the set period expires by taking not only the information about the current state of the user U1 and the target health condition of the user U1 after the lapse of the set period but also the budget of the user into consideration.

Note that the health management support system 1 may be configured so as to be able to acquire the progress of the behavioral plan by the user U1 through the operation terminal 20 or a scale or the like that can communicate with the health management support system 1. In this way, the health management support system 1 can calculate and present the achievement level of the adopted behavioral plan based on the acquired progress of the behavioral plan by the user U1, and can propose, when the achievement level is low, revised behavioral items for those originally included in the behavioral plan.

Further, the health management support system 1 may be configured so as to propose a behavioral plan for the user U1 by referring to behavioral items and improvements in health conditions, based on a selected plan (an adopted behavioral plan), of a plurality of past users. For example, at least behavioral items and improvements in health conditions, based on a selected plan, of a plurality of past users (past subjects) are registered in a first database. The plan calculation unit 12 extracts a behavioral item and an improvement in a health condition, based on the selected plan, of at least one subject of which the information about the state at that time, the target health condition after the lapse of the set period, and the budget have been determined to be similar to the information about the current state, the target health condition after the lapse of the set period, and the budget of the user U1. Then, the plan calculation unit 12 calculates at least one behavioral plan for the user U1 by referring to the behavioral item and the improvement in the health condition based on the extracted selected plan. In this way, the health management support system 1 can propose a more realistic behavioral plan.

### (Operation of Health Management Support System 1)

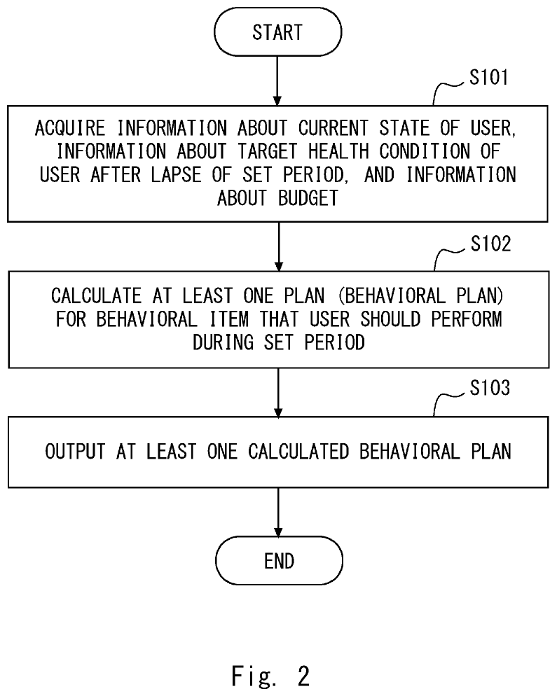

Next, operations performed by the health management support system 1 will be described with reference to FIG. 2. FIG. 2 is a flowchart showing operations performed by the health management support system 1.

Firstly, the operation terminal 20 receives information about the current state of the user U1, information about the target health condition of the user U1 after the lapse of the set period, and information about the budget, entered by an operation performed by the user U1, and transmits the received information items to the health management support apparatus 10 through the network 50.

Note that as described above, in this embodiment, the monitor 30 is used as the monitor of the operation terminal 20, which is a PC terminal. Therefore, input items for the information about the current state of the user U1 and the like are displayed on the monitor 30.

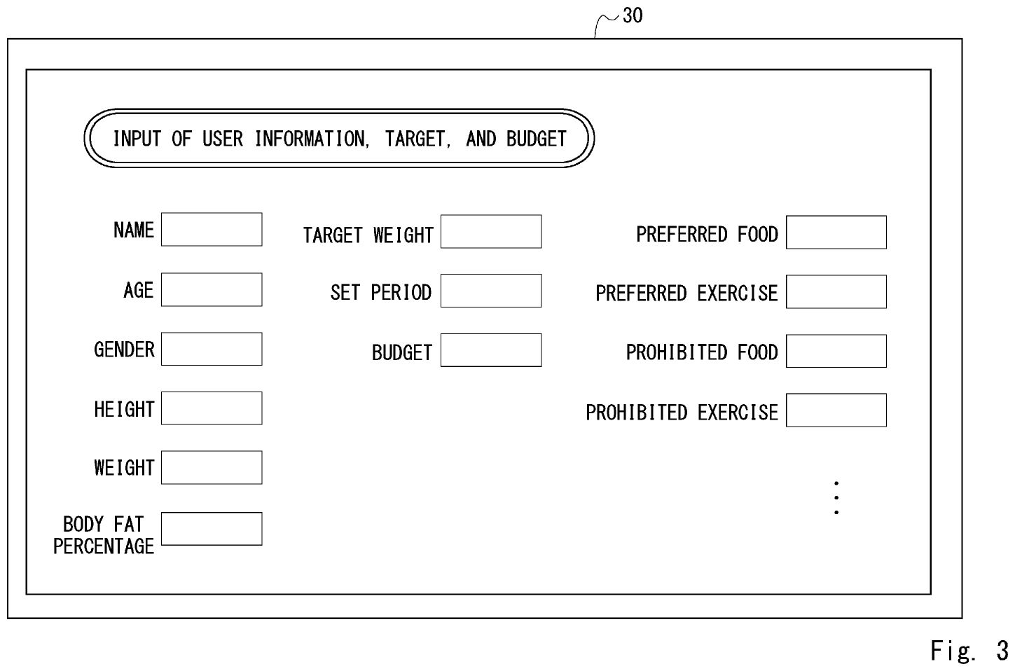

FIG. 3 shows a list of input items displayed on the monitor 30. As shown in FIG. 3, as the input items for the information about the current state of the user U1, input items such as the name, the age, the gender, the height, the weight, and the body fat percentage of the user U1 are displayed on the monitor 30. Note that the body fat percentage may be directly entered by the user U1 or automatically calculated from the height, the weight, and the like. Further, as the input items for the information about the target health condition of the user U1 after the lapse of the set period, input items such as a target weight and a set period are also displayed on the monitor 30. Further, an input item for the budget is also displayed on the monitor 30. Further, input items such as a preferred food, a preferred exercise, a prohibited food, and a prohibited exercise are also displayed on the monitor 30.

For example, the user U1 enters necessary information in input items displayed on the monitor 30 by operating a keyboard or the like of the operation terminal 20, which is a PC terminal, and thereby enters information about the current state of the user U1, information about the target health condition of the user U1 after the lapse of the set period, and information about the budget of the user into the operation terminal 20. The operation terminal 20 receives these information items entered by the operation performed by the user U1, and transmits the received information items to the health management support apparatus 10 through the network 50.

In the health management support apparatus 10, the information acquisition unit 11 acquires the information entered by the user U1 through the operation terminal 20. That is, the information acquisition unit 11 acquires, for example, the information about the current state of the user U1, the information about the target health condition of the user U1 after the lapse of the set period, and the information about the budget (Step S101).

After that, in the health management support apparatus 10, plan calculation unit 12 calculates, based on the information about the current state of the user U1, the target health condition of the user U1 after the lapse of the set period, and the budget, acquired by the information acquisition unit 11, at least one plan (one behavioral plan) for behavioral items that the user U1 should perform during the set period (Step S102).

For example, as the information about the current state of the user U1, the current weight of the user U1 is set to 80 kg. Further, regarding the target health condition of the user U1 after the lapse of the set period, the set period is set to three months and the target weight of the user U1 after the lapse of the set period is set to 70 kg. Furthermore, the budget is set to 50,000 yen. In this case, the plan calculation unit 12 calculates at least one behavioral plan including meals and exercise items for reducing the weight of the user U1 from 80 kg to 70 kg in three months.

After that, the output unit 13 outputs at least one behavioral plan calculated by the plan calculation unit 12 (Step S103). The information about at least one behavioral plan output from the output unit 13 is transferred, for example, to the monitor 30 through the network 50, and is displayed on the monitor 30.

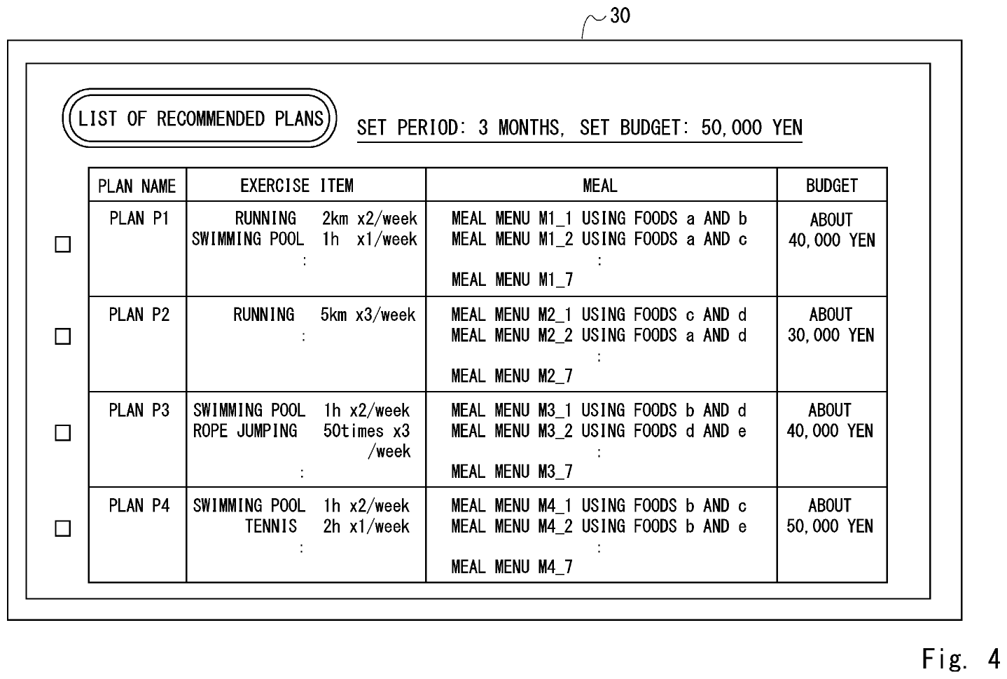

FIG. 4 shows an example of behavioral plans displayed on the monitor 30. As shown in FIG. 4, four behavioral plans P1 to P4 are displayed on the monitor 30 as behavioral plans (recommended plans) proposed to the user U1. Note that the budgets of the four behavioral plans P1 to P4 are all equal to or lower than 50,000 yen as set by the user U1.

In the behavioral plan P1, it is proposed to incorporate, as exercise items, two 2 km runs and one one-hour (swimming) session in a swimming pool in each week. Further, in the behavioral plan P1, it is proposed to incorporate, as meals, meal menus M1_1 to M1_7 including a meal menu M1_1 using foods a and b, and a meal menu M1_2 using foods a and c in each week. The budget is about 40,000 yen.

In the behavioral plan P2, it is proposed to incorporate, as exercise items, three 5 km runs in each week. Further, in the behavioral plan P2, it is proposed to incorporate, as meals, meal menus M2_1 to M2_7 including a meal menu M2_1 using foods c and d, and a meal menu M2_2 using foods a and d in each week. The budget is about 30,000 yen.

In the behavioral plan P3, it is proposed to incorporate, as exercise items, two one-hour swimming sessions in a swimming pool and three 50 rope jumping sessions in each week. Further, in the behavioral plan P3, it is proposed to incorporate, as meals, meal menus M3_1 to M3_7 including a meal menu M3_1 using foods b and d, and a meal menu M3_2 using foods d and e in each week. The budget is about 40,000 yen.

In the behavioral plan P4, it is proposed to incorporate, as exercise items, two one-hour swimming sessions in a swimming pool and one two-hour tennis practices in each week. Further, in the behavioral plan P4, it is proposed to incorporate, as meals, meal menus M4_1 to M4_7 including a meal menu M4_1 using foods b and c, and a meal menu M4_2 using foods b and e in each week. The budget is about 50,000 yen.

For example, the user U1 may adopt (i.e., choose) one of the behavioral plans P1 to P4 that he/she likes, and behaves while incorporating the meals and the exercise items proposed in the adopted behavioral plan. By doing so, the user U1 can bring his/her health condition (his/her weight in this example) closer to the target health condition before the set period of three months expires within the set budget. That is, the health management support system 1 can propose an appropriate behavioral plan for bringing the health condition of the user U1 closer to his/her target health condition before the set period expires within the budget.

Note that more specific items or the like may be presented (i.e., shown) as exercise items and meals included in each behavioral plan. For example, instead of the “one-hour swimming session in a swimming pool”, “20 times of 50 m crawls” or the like may be presented in a more specific manner. Alternatively, for example, instead of the “one-hour gym training session”, “three sets of 10 times of kg bench press, three sets of 10 times of 50 kg leg press, . . . .” or the like may be presented in a more specific manner.

Further, although a case where the objective is to reduce the weight has been described in the example shown in FIG. 4, the objective is not limited to this example. For example, the objective may be to reduce the body fat percentage or to change the body shape to a desired body shape. Note that in the case where the objective is to change the body shape to a desired body shape, the health management support system 1 may be configured so as to be able to acquire information about the body shape of the user U1, for example, from an image of the user U1 taken by a camera that can communicate with the health management support system 1.

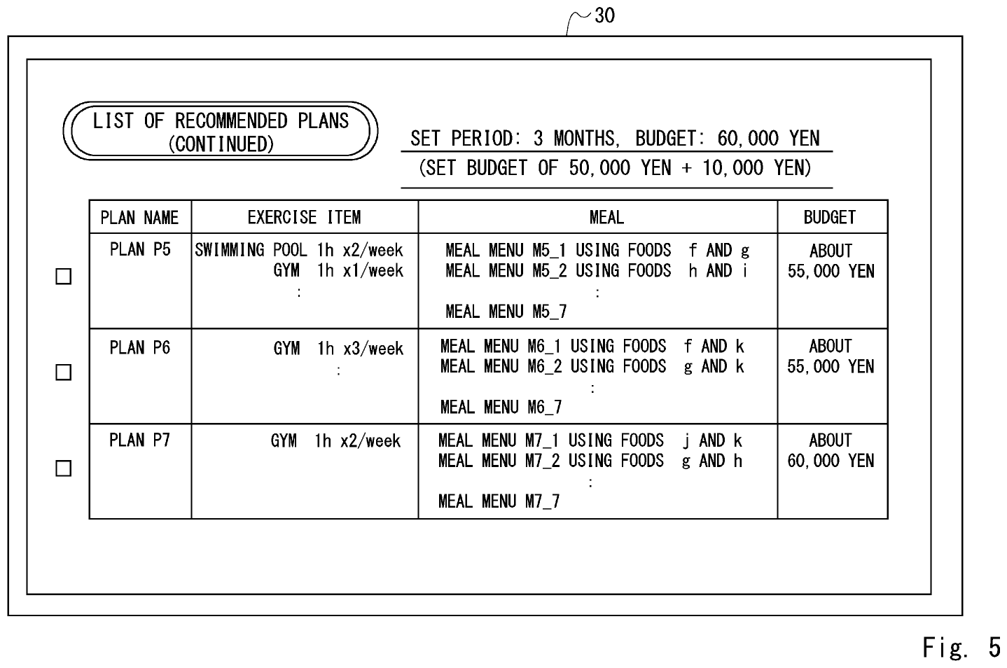

Further, although a case where the behavioral plans P1 to P4 that can be carried out within the set budget has been described in the example shown in FIG. 4, the present disclosure is not limited to this example. For example, a behavioral plan that can be carried out within a budget that is a predetermined amount larger than the set budget may also be proposed. That is, the health management support system 1 may be configured to so as to be able to also calculate and output a behavioral plan that can be carried out within a budget that is a predetermined amount larger than the set budget. This feature will be briefly described hereinafter with reference to FIG. 5.

FIG. 5 shows another example of a behavioral plan displayed on the monitor In the example shown in FIG. 5, three behavioral plans P5 to P7 that can be carried out within a budget that is a predetermined amount larger than the set budget. In the example shown in FIG. 5, the behavioral plans P1 to P4 that can be carried out within the set budget are omitted. Note that the budgets for the three behavioral plans P5 to P7 are equal to or lower than 60,000 yen, which is 10,000 yen larger than the budget set by the user U1.

In the behavioral plan P5, it is proposed to incorporate, as exercise items, two one-hour (swimming) sessions in a swimming pool and one one-hour training in a gym (e.g., a strength training using a machine) in each week. Further, in the behavioral plan P5, it is proposed to incorporate, as meals, meal menus M5_1 to M5_7 including a meal menu M5_1 using foods f and g, and a meal menu M5_2 using foods h and i in each week. The budget is about 55,000 yen.

In the behavioral plan P6, it is proposed to incorporate, as exercise items, three one-hour trainings in a gym in each week. Further, in the behavioral plan P6, it is proposed to incorporate, as meals, meal menus M6_1 to M6_7 including a meal menu M6_1 using foods f and k, and a meal menu M6_2 using foods g and k in each week. The budget is about 55,000 yen.

In the behavioral plan P7, it is proposed to incorporate, as exercise items, two one-hour trainings in a gym in each week. Further, in the behavioral plan P7, it is proposed to incorporate, as meals, meal menus M7_1 to M7_7 including a meal menu M7_1 using foods j and k, and a meal menu M7_2 using foods g and h in each week. The budget is about 60,000 yen.

In this way, for example, even when the user U1 cannot find a preferred behavioral plan in any of the behavioral plans P1 to P4 that can be carried out within the set budget, the user U1 can adopt (i.e., choose) one of the behavioral plans P5 to P7 that he or she likes, provided that the user U1 can accept a budget that is slightly higher than the set budget.

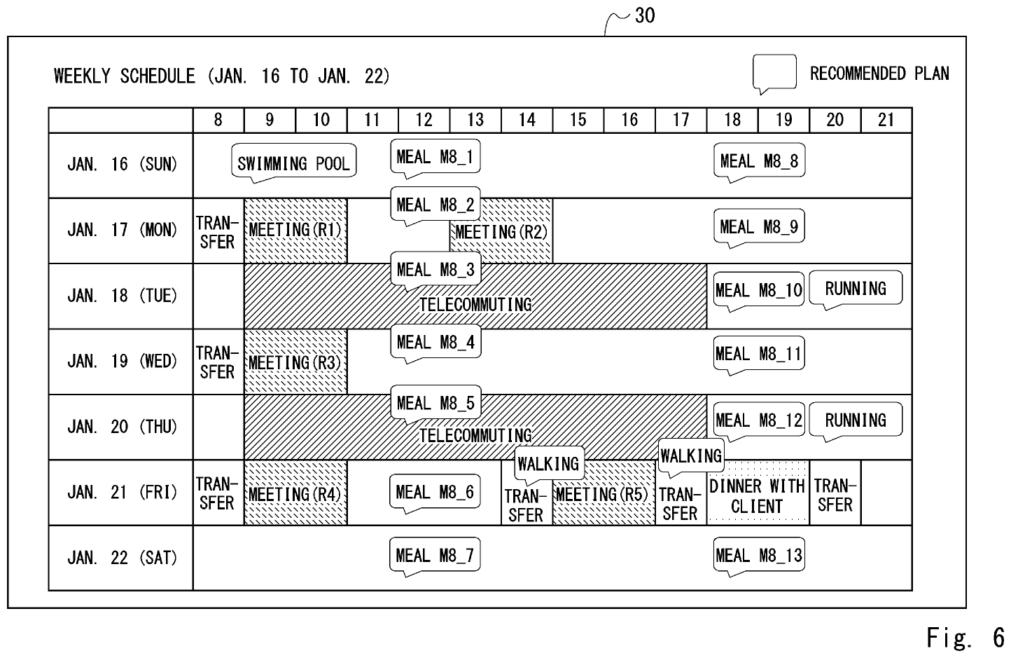

Further, the health management support system 1 may be configured to calculate and propose a behavioral plan for the user U1 while also taking a work schedule or the like of the user U1 into consideration. Note that the schedule of the user U1 is registered, for example, in a second database. That is, the health management support system 1 may be configured to calculate a behavioral plan while also taking, for example, a spare time of the user U1, calories that will be taken in a dinner with a client or the like, and/or calories that will be consumed by commuting, a physical work, or the like into consideration. The behavioral plan calculated in the above-described calculation is displayed, for example, on the monitor 30 along with the schedule of the user U1. This feature will be briefly described hereinafter with reference to FIG. 6.

FIG. 6 shows an example of a behavioral plan displayed on the monitor 30 along with a work schedule of a user. In the example shown in FIG. 6, commuting to a workplace is scheduled on January 17, 19 and 21, and telecommuting is scheduled on January 18 and 20. Further, a meeting that will be held at a client company or the like is scheduled in the afternoon of January 21, and a subsequent dinner with the client or the like is also scheduled. In this case, the health management support system 1 calculates the behavioral plan while also taking calories that will be taken in the dinner, calories that will be consumed by the commuting, and calories that will be consumed by the traveling between the workplace and the client company or the like into consideration. In the behavioral plan shown in FIG. 6, it is proposed to incorporate, as meals, lunch menus M8_1 to M8_7 and dinner menus M8_8 to M8_13 for the days except for the day on which the dinner with the client or the like will be held, in each week. Note that, for example, dishes and the like in menus in a canteen of the own company or the like may be used for the lunch menus. Further, it is proposed to incorporate, as exercise items, a (swimming) session in a swimming pool in the spare time in the morning of January 16 and running at nights of January 18 and 20. Further, it is proposed to walk from the workplace to the client company or the like and from the client company or the like to the place where the dinner will be held.

In this way, the health management support system 1 can propose a more appropriate behavioral plan without affecting the already fixed schedule of the user U1 and while taking the effect of the already fixed schedule of the user U1 into consideration.

As described above, the health management support system 1 according to this embodiment can propose an appropriate behavioral plan for bringing the health condition of the user U1 closer to his/her target health condition before the set period expires by taking not only the information about the current state of the user U1 and the target health condition of the user U1 after the lapse of the set period but also the budget of the user into consideration.

Note that the present disclosure is not limited to the above-described embodiments, and they can be modified as appropriate without departing from the scope and spirit of the disclosure.

Further, in the present disclosure, some or all of the control processes performed in the health management support system 1 can be implemented by having a CPU (Central Processing Unit) execute a computer program.

In the aforementioned program includes instructions (or software codes) that, when loaded into a computer, cause the computer to perform at least one of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A health management support system comprising:
an acquisition unit configured to acquire information about a current state of a user, information about a target health condition of the user after a lapse of a set period, and information about a budget of the user;
a plan calculation unit configured to calculate at least one plan for a behavioral item that the user should perform during the set period based on the information about the current state of the user, the target health condition of the user after the lapse of the set period, and the budget; and
an output unit configured to output the at least one plan calculated by the plan calculation unit.

2. The health management support system according to claim 1, wherein the information about the current state of the user includes information about a current health condition of the user.

3. The health management support system according to claim 1, wherein the information about the current state of the user includes information about at least one of a current age, a gender, a current height, and a current weight of the user.

4. The health management support system according to claim 1, wherein the information about the current state of the user includes information about at least one of a currently preferred food of the user and a currently preferred exercise thereof.

5. The health management support system according to claim 1, wherein the information about the current state of the user includes at least one of information about a food that the user is currently prohibited from ingesting and information about an exercise that the user is currently prohibited from doing.

6. The health management support system according to claim 1, wherein the behavioral item of the user includes at least one of an exercise item and a meal.

7. The health management support system according to claim 1, wherein
the plan calculation unit:
extracts a behavioral item and an improvement in a health condition, based on a selected plan, of at least one past subject of which, among behavioral items and improvements in health conditions, based on the selected plan, of a plurality of past subjects registered in a first database, the information about the state at that time, the target health condition after the lapse of the set period, and the budget have been determined to be similar to the information about the current state, the target health condition after the lapse of the set period, and the budget of the user; and
calculates at least one plan for a behavioral item that the user should perform during the set period by referring to the behavioral item and the improvement in the health condition, based on the selected plan, of the extracted past subject.

8. The health management support system according to claim 1, wherein
the plan calculation unit also calculates at least one new plan for the behavioral item that the user should perform during the set period based on a new budget that is obtained by adding a predetermined amount of money to the budget, and
the output unit also outputs the at least one new plan calculated by the plan calculation unit along with information about the new budget.

9. The health management support system according to claim 1, wherein the plan calculation unit calculates, in addition to the information about the current state of the user, the target health condition of the user after the lapse of the set period, and the budget, at least one plan for the behavioral item that the user should perform during the set period based on a schedule of the user during the set period stored in a second database.

10. The health management support system according to claim 1, wherein the output unit displays one of selected plans among the at least one plan calculated by the plan calculation unit on a display unit along with the schedule of the user.

11. A method for controlling a health management support system, comprising:
acquiring information about a current state of a user, information about a target health condition of the user after a lapse of a set period, and information about a budget of the user;
calculating at least one plan for a behavioral item that the user should perform during the set period based on the information about the current state of the user, the target health condition of the user after the lapse of the set period, and the budget; and
outputting the at least one plan calculated by the plan calculation unit.

12. A non-transitory computer readable storage medium storing a control program for causing a computer to perform:
a process for acquiring information about a current state of a user,
information about a target health condition of the user after a lapse of a set period, and information about a budget of the user;
a process for calculating at least one plan for a behavioral item that the user should perform during the set period based on the information about the current state of the user, the target health condition of the user after the lapse of the set period, and the budget; and
a process for outputting the at least one plan calculated by the plan calculation unit.
